(19) United States
(12) Patent Application Publication
(10) Pub. No.: US 2016/0338942 A1
(43) Pub. Date: Nov. 24, 2016

(54) Get faded customized shaving lotion

(71) Applicant: Melissa Smith, Las Cruces, NM (US)

(72) Inventor: Melissa Smith, Las Cruces, NM (US)

(21) Appl. No.: 14/544,878

(22) Filed: May 20, 2015

(51) Int. Cl. A61K 8/97; A61Q 9/02; A61K 8/92

(52) U.S. Cl. CPC A61K 8/97; A61K 8/922; A61Q 9/02

(57) ABSTRACT

Begin by trimming excess hair from the top of the customers head with clippers using the blade size determined by the customers preferences; rinse the customers head with warm water in order to remove excess hair and open up pores, which allows for a closer shave; apply the invented Get Faded Barbershop & Salon's custom shaving lotion (ingredients previously provided); apply either choice of hot lather or cold shaving cream to the customers head; shave with a disposabe razor the sides of the customers head using a upward, downward, and side to side motion, re-rinse the customers head to remove any excess hair, do a second application of the custom shaving lotion; finalize by tracing over fade line with 000 blade in order to blend, go over blended fade line with clippers, go around the head with electric shaver. remove any stray or excess hairs with fade brush.

This product could be used for any part of the body to help alleviate razor burn or bumps and helps stun hair growth; which keeps a closer clean shave longer.

Ingredients
8 oz. Image Essentials Aloe Vera
2 oz. Family Dollar Vitamin E oil with soybean oil and coconut oil
2 oz. Aloe Farms Aloe Vera Gel

### Directions

1. Combine all ingredients in a medium sized dispenser bottle.

2. Shake well

3. refrigerate overnight

4. Remove bottle from refrigerator shake for five minutes

5. It is now ready for use

1. For Get Faded customized shaving lotion:
A moisturizing product compromising:
(a) A product that when used applied directly on skin alleviates razor burn and bumps,
(b) Such product also stuns hair growth,
(c) Stated product can be used on any part of the body that hair needs to be removed from.

2. For the production of Get Faded customized shaving lotion:
(a) In a 32 oz. bottle add 24 oz. of Aloe Vera gel
(b) Add 4 oz. Vitamin E oil-enriched with coconut oil,
(c) Add 1 oz. Aloe Farms, aloe Vera gel;
(d) Shake the bottle for 5 minutes,
(e) Freeze the bottle for 2 hours
(f) Shake again after the bottle has defrosted.

3. For using the Get Faded customized shaving lotion:
A process for a hot or cold lather razor fade to the head of a customer, which compromises the steps of:
(a) Trimming hair using a 00000 blade with hair clippers on the sides of the head,
(b) Trim hair on the top of the head to the desired height,
(c) Rinse the head with warm water,
(d) Apply the Get Faded customized shaving lotion,
(e) Apply hot or cold lather to the head,
(f) Using a disposable razor shave the sides of head in all directions,
(g) Re-rinse head to remove all excess hair,
(h) Apply a second application of the Get Faded customized shaving lotion,
(i) Trace over fade line with 000 blade,
(j) Go over fade line with clippers,
(k) Using an electric shaver go around the head,
(l) Finalize by brushing off any access hairs.
